(19) United States
(12) Patent Application Publication
(10) Pub. No.: US 2010/0144802 A1
(43) Pub. Date: Jun. 10, 2010

(54) Small Molecule Inhibitors for Immune Modulation

(76) Inventors: Amy B. Heimberger, Houston, TX (US); Waldemar Priebe, Houston, TX (US); Izabela Fokt, Houston, TX (US); Slawomir Szymanski, Houston, TX (US); Sakina Hussain, Houston, TX (US); Ling Y. Kong, Houston, TX (US)

Correspondence Address: NIELSEN IP LAW LLC, 1177 West Loop South, Suite 1600, Houston, TX, 77027, US

(21) Appl. No.: 12/593,280

(22) PCT Filed: Mar. 28, 2008
(86) PCT No.: PCT/US08/58727

§ 371 Date: Jan. 19, 2010

Related U.S. Application Data
(60) Provisional application No. 60/908,559, filed on Mar. 28, 2007.

(51) Int. Cl. A61K 31/44; A61P 37/04

(52) U.S. Cl. 514/357

(57) ABSTRACT

Methods of treating immunosuppression by administering a therapeutic amount of a tryphostin compound of the formula:

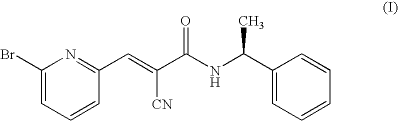

### CROSS-REFERENCE TO RELATED APPLICATIONS

This application claims the benefit of U.S. Provisional Application Ser. No. 60/908,559 filed on Mar. 28, 2007, which is incorporated by reference.

### STATEMENT REGARDING FEDERALLY SPONSORED RESEARCH OR DEVELOPMENT

MDACC Start-up Funds, MDA Acct. No. 179786. NIH Modulation of Microglia and T Cell Interactions in Malignant Glioma 1 RO1 CA120813-01A1, No. CA120813-01A1.

### FIELD OF THE INVENTION

The present invention is generally related to treating immunological suppression, and more particularly directed to methods of inducing immunological responses by administering tryphostin and tryphostin-like compounds.

### THE NAMES OF THE PARTIES TO A JOINT RESEARCH AGREEMENT

None.

### REFERENCE TO SEQUENCE LISTING

None.

### BACKGROUND OF THE INVENTION

Deficient immune responses play a critical role in the dismal prognosis of malignant glioma patients and patients with other cancers. Patients with malignant human gliomas have a preponderance of immunosuppressive cytokines, a lack of effector/activated T cells, enhanced regulatory T cells (Tregs), and inadequate antigen-presentation functional activity, each contributing to profound immunosuppression. Known natural immune stimulators within these patients are insufficient to overcome these influences. Recently reported, microglia/macrophages isolated from human gliomas were found lacking the expression of the costimulatory molecules CD86 and CD80 and were unable to activate naive T cells. Hussain, S. F. et al. Neuro-oncol 8, 261-279 (2006). In addition, in cancer cells, the Src family of kinases, a family of non-receptor tyrosine kinases have been found to be involved in the signal transduction. The Src family of kinases (“SFKs”) are a family of non-receptor tyrosine kinases that are involved in signal transduction in cancer cells.

SFKs and certain growth factor receptors are overexpressed in various cancers. Halpern M. S., England J. M., Kopen G. C., Christou A. A., Taylor R. L. Jr., Endogenous c-src as a Determinant of the Tumorigenicity of src Oncogenes, Proc Natl Acad Sci USA. 1996 93(2): 824-827. Haura, E. B., Zheng, Z., Song, L., Cantor, A., Bepler, G., Activated Epidermal Growth Factor Receptor-Stat-3 Signaling Promotes Tumor Survival In Vivo in Non-Small Cell Lung Cancer, Clin. Cancer Res. 2005, 11(23): 8288-8294. Likewise, the activation paradigm and role of STATs (signal transducers and activators of transcription proteins) in certain cancers has been reported. See Yu, H., Jove, R., The Stats of Cancer—New Molecular Targets Come of Age, Nature Rev. 2004, 4: 97-106.

A role for SFKs in the initiation and/or progression of cancer has been demonstrated in multiple tumor cell lines. Id.; See also, Trevino, J. G., Summy, J. M., Lesslie, D. P., Parikh, N. U., Hong, D. S., Lee, F. Y., Donato, N. J., Abbruzzese, J. L., Baker, C. H., and Gallick, G. E., Inhibition of SRC Expression and Activity Inhibits Tumor Progression and Metastasis of Human Pancreatic Adenocarcinoma Cells in an Orthotopic Nude Mouse Model. Am J Pathol, 168: 962-972, 2006. For example, in epithelial cancers, SFKs facilitate epithelial-to-mesenchymal transition, which may be important in cancer progression. See e.g., Johnson, F. M. and Gallick, G. E., Src Family of Non-Receptor Tyrosine Kinases as Molecular Targets for Cancer Therapy. Current Medicinal Chemistry, In Press, 2006.

At least one member of the Src family of kinases (SFKs), c-Src, reportedly induces STATs involved in the tumorigenesis process. Xi, S., Zhang, Q., Dyer, K. F., Lerner, E. C., Smithgall, T. E. Gooding, W. E., Kamens, J., Grandis, J. R., Src Kinases Mediate STAT Growth Pathways in Squamous Cell Carcinoma of the Head and Neck, J. Biol. Chem. 2003, 278(34): 31574-31583. In particular, STAT3 is a member of the signal transducer and activator of transcription protein family that regulates many aspects of cell growth, survival and differentiation. Constitutive STAT3 has been associated with various human cancers and commonly suggests poor prognosis as it has anti-apoptotic as well as proliferative effects. Yu, H. and Jove, R. The STATs of Cancer—New Molecular Targets Come of Age, Nat Rev Cancer, 4: 97-105, 2004. Src family kinases (SFK) also mediate STAT growth pathways in various cancers. Xi, S., Zhang, Q., Dyer, K. F., Lerner, E. C., Smithgall, T. E., Gooding, W. E., Kamens, J., and Grandis, J. R., Src kinases Mediate STAT Growth Pathways in Squamous Cell Carcinoma of the Head and Neck, J Biol Chem, 278: 31574-31583, 2003. Reportedly, in normal peripheral blood mononuclear cells (PBMCs), when Stat-3 was blocked, macrophages increased production of IL-12, induced Th1 responses, and reversed systemic tolerance. Cheng, F. et al. Immunity 19, 425-36 (2003).

Kinase inhibitors that inhibit the JAK2/Stat3 signal have potential as anti-cancer drugs. However, patients with malignant human gliomas need to attenuate suppressed immune responses. A need exists, therefore, for therapeutics that exhibit strong anti-proliferative effects on cancer while activating potent immune responses to treat the immunosuppressed cancer patient.

### BRIEF SUMMARY OF THE INVENTION

The present invention provides methods of induce immunostimulatory responses including immunostimulatory cytokines, costimulatory molecules, and intracellular signaling in macrophages, and also enhance phosphorylation of key signaling molecules (ZAP-70, Lck) and effector function of T cells in these patients. The methods of the subject invention demonstrate novel immunotherapeutic function for various compounds in the treatment of immunosuppressed cancer patients.

### BRIEF DESCRIPTION OF THE SEVERAL VIEWS OF THE DRAWINGS

The foregoing and other features and aspects of the present invention will be best understood with reference to the following detailed description of a specific embodiment of the invention, when read in conjunction with the accompanying drawings, wherein:

FIG. 1A is the tryphostin compound (WP1066) useful in connection with the present invention.

FIG. 1B shows that WP1066 can cross blood brain barrier.

FIG. 1C shows that WP1066 inhibits Stat-3.

FIG. 1D shows that cell viability is not comprised when WP1066 inhibits STAT3.

FIG. 2A shows that WP1066 is capable of upregulating CD80 and CD86.

FIG. 2B shows that WP1066 can induce crucial cyokines that stimulate T cell effector function.

FIG. 2C shows that WP1066 can restore T cell responsiveness.

FIG. 2D:a shows the effect of WP1066 on GBM patient monocyte.

FIG. 2D:b shows the effect of WP1066 on GBM patient T cells.

FIG. 3A shows survival data from C57BL/6J mice treated with WP1066 after intracerebral B16EGFRvIII cells were established in the brain.

FIG. 3B shows survival data from nude mice treated with WP1066 after intracerebral B16 were established in the brain.

FIG. 4 shows that WP1066 enhances immune cytotoxicity but not humoral responses.

FIG. 5 shows that WP1066 enhances immune cytotoxicity but not humoral responses.

FIG. 6 shows that WP1066 inhibits Foxp3 induction in peripheral T cells and downregulates Foxp3 expression in natural Tregs.

### DETAILED DESCRIPTION OF THE INVENTION

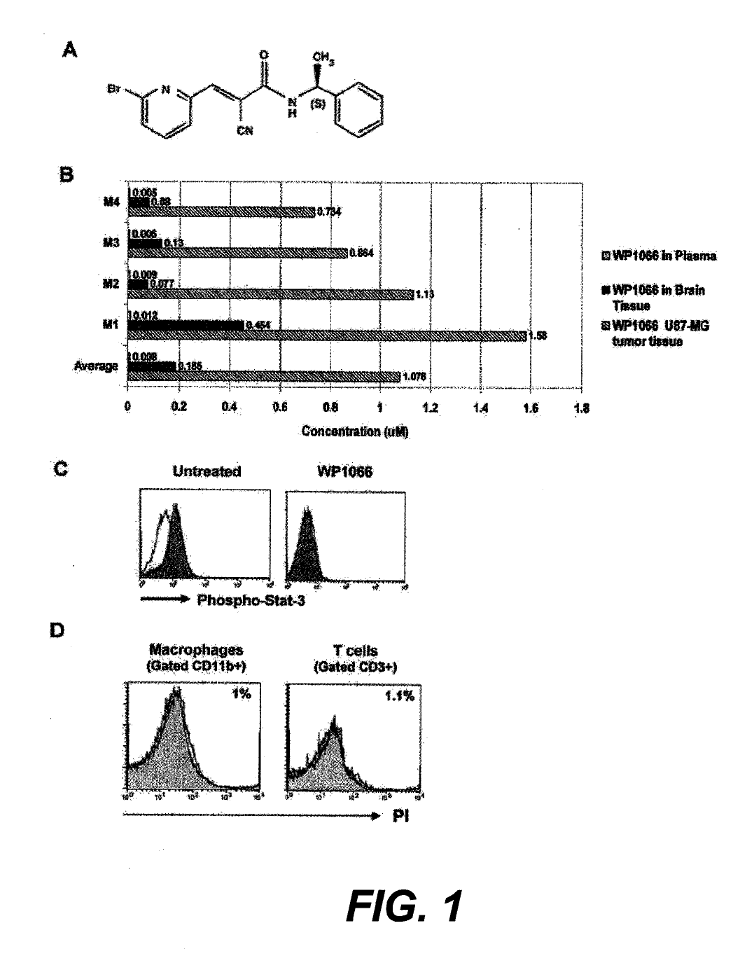

Patients with malignant human gliomas have a preponderance of immunosuppressive cytokines, a lack of effector/activated T cells, enhanced regulatory T cells (Tregs), and inadequate antigen-presentation functional activity, contributing to their profound immunosuppression. Known immune stimulators within these patients are insufficient to overcome these influences. Therefore, we investigated whether a pharmacological derivative of the natural compound, caffeic acid (WP1066)(FIG. 1A), a small molecule inhibitor of Stat-3 activity, can (in physiologically relevant doses) attenuate these suppressed immune responses. In mice, plasma concentrations of >2 μM are achieved via oral administration, and WP1066 can effectively cross the blood-brain barrier and achieve central nervous system (CNS) concentrations of 0.185 μM and within orthotopic gliomas concentrations of 1.07 μM, a critical factor when determining therapies for glioma patients (FIG. 1B).

Stat-3 is constitutively activated in many tumors, and WP1066 has been shown to inhibit Stat-3 and have proapoptotic activity in a variety of tumor cell types, including malignant gliomas. Weissenberger, J. et al. Oncogene 23, 3308-16 (2004); Iwamaru, A. et al. Oncogene published online 16 Oct. 2006 (doi: 10.1038sj.onc.1210031). The proapoptotic activity WP1066 possesses may contribute to an induced neutropenia and lymphopenia, but at doses achievable in vivo (1 nM-5 μM) that inhibit Stat-3 within immune cells (FIG. 1C), cell viability is not compromised (FIG. 1D), suggesting that WP1066 could be employed as an immune adjuvant.

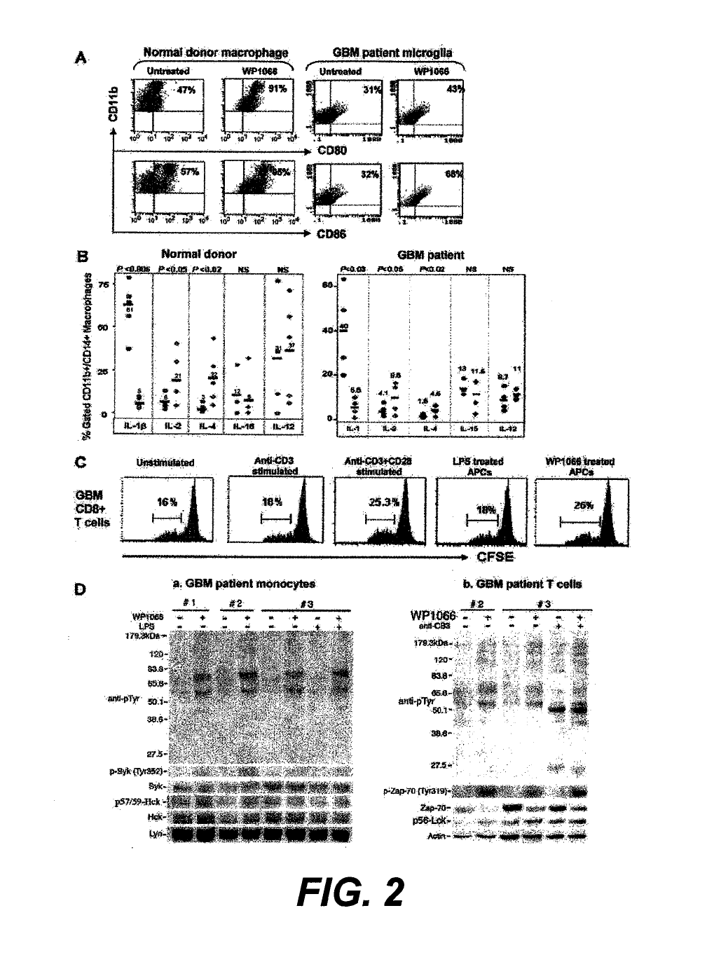

Microglia/macrophages isolated from human gliomas lack the expression of the costimulatory molecules CD86 and CD80 and are unable to activate naive T cells. Hussain, S. F. et al. Neuro-oncol 8, 261-279 (2006). In normal peripheral blood mononuclear cells (PBMCs), when Stat-3 was blocked, macrophages increased production of IL-12, induced Th1 responses, and reversed systemic tolerance. Cheng, F. et al. Immunity 19, 425-36 (2003). However, WP1066 and other similar tyrphostin and tyrphostin-like compounds disclosed in US 2005/0277680, at paragraphs 0009 through 0047 and paragraphs 0079 through 0117, incorporated herein by reference, are capable of upregulating CD80 and CD86 on both normal donor PBMCs and also on tumor-infiltrating microglia/macrophages freshly isolated from glioblastoma multiforme (GBM) patients (FIG. 2A). This up regulation of costimulation can be achieved when human monocytes are incubated with the immunosuppressive cytokines IL-10 and TGF-β (data not shown). Furthermore, known potent immune activators such as lipopolysaccharide (LPS) were totally insufficient in up regulating these costimulatory on microglia isolated directly from GBM patients, indicating that WP1066 is a potent agent for inducing costimulation. Hussain, S. F. et al. Neuro-oncol 8, 261-279 (2006).

Human glioma patients have also been shown to be deficient in immunostimulatory cytokines. Heimberger, A. B., Bigner, D. D. & Sampson, J. J. In Brain Tumor Immunotherapy (eds. Liau, L. M., Becker, D. P., Cloughesy, T. F. & Bigner, D. D.) 101-130 (Humana Press Inc., Totowa, N. J., 2000). WP1066 can induce the crucial cytokines that stimulate T cell effector function such as IL-2, IL-4, IL-12, and IL-15 (FIG. 2B) from macrophages isolated from normal donors and from GBM patients. IL-2 and IL-15 have been used successfully in cancer immunotherapy to induce proliferation of tumor-infiltrating lymphocytes. Rosenberg, S. A. Cancer J Sci Am 6 Suppl 1, S2-7 (2000); Waldmann, T. A. Nat Rev Immunol 6, 595-601 (2006). IL-4 induces antitumor factors and can cause antigen-presenting-cell (APC) differentiation characterized by efficient antigen uptake and processing. Hung, K. et al. J Exp Med 188, 2357-68 (1998); Pardoll, D. M. Nat Rev Immunol 2, 227-38 (2002). Although LPS induced significantly higher levels of the inflammatory cytokine IL-1β compared with WP1066, IL-1 has also been shown to promote tumor cell growth. Saijo, Y. et al. J Immunol 169, 469-75 (2002). This data indicates that WP1066 is capable of inducing a proinflammatory cytokine cascade.

Furthermore, T cells isolated from peripheral blood of GBM patients and incubated with allo-CD11b⁺ macrophages are impaired in their proliferative response to anti-CD3 stimulation. When additional costimulation is provided with anti-CD28 antibody, T cell proliferation is augmented in these patients. Furthermore, LPS-treated macrophages were unable to induce any T cell proliferation. In contrast to LPS, physiological doses of WP1066 potently induced T cell proliferation without additional co-stimulation (FIG. 2C), indicating that WP1066 can restore T cell responsiveness known to be profoundly impaired in GBM patients.

The invention is further illustrated by the following examples.

### Example 1

To determine how WP1066 induces these potent immune responses, T cells and monocytes isolated from GBM patients were treated with WP1066 and subsequently lysed and analyzed by immunoblotting for critical signaling proteins. In contrast to LPS or anti-CD3 antibody, WP1066 induced or enhanced tyrosine phosphorylation in both monocytes and T cells, producing the most striking increase levels of proteins of 70 to 75 KDa and 50 to 60 KDa (FIG. 2D). Further analysis revealed that WP1066 induced phospho-p72Syk (Tyr352) and phospho-ZAP-70 (Tyr319) in monocytes and T cells, respectively. WP1066 also increased levels of the Src family protein tyrosine kinase p57/p59-Hck in monocytes and p56-Lck in T cells (FIG. 2D). The tyrosine phosphorylation of Syk initiates downstream signaling events during human monocyte activation, whereas the phosphorylation of Tyr319 in Zap-70 plays a critical role in mediating T cell activation via signaling through the T cell receptor. Raeder, E. M. et al. J Immunol 163, 6785-93 (1999); Williams, B. L. et al. EMBO 18, 1832-1844 (1999). Both Syk and ZAP-70 can be phosphorylated through autophosphorylation and transphosphorylation. Although WP1066 didn't affect the total protein level of Syk (FIG. 2D:a), it markedly decreased the total protein level of ZAP-70 (FIG. 2D:b), suggesting that WP1066 may change localization and/or stability of ZAP-70. The tyrosine phosphorylation of critical signaling proteins induced/enhanced by WP1066 are likely pivotal to the WP1066-induced restoration of immune function in GBM patients and explains the mechanisms of potent immune activation.

FIG. 1 depicts the novel small molecule inhibitor, WP1066, that inhibits Stat-3 activity and has significant central nervous system (CNS) and glioma tumor penetration in vitro and in vivo. FIG. 1A provides the chemical structure of WP1066. FIG. 1B shows the administration of WP1066 resulted in significant CNS penetration and accumulation within malignant gliomas. WP1066 was injected intraperitoneally (IP) at doses of 100 mg/kg every other day for two weeks in nude mice. Specifically, plasma, CNS tissue, and U87-MG flank tumors were harvested and, after extraction, were analyzed for WP1066 content using tandem liquid chromatography/mass spectrometry. WP1066 has a plasma half-life of >4 hours in this murine model, and studies in the animals bearing flank tumors demonstrated selective uptake of the compound by tumors, which showed a higher drug content than any other tissue. WP1066 delivered IP at doses of 100 mg/kg every other day for up to two weeks or intravenously at doses of only 10 mg/kg (data not shown) achieved plasma concentrations in excess of 1 μM, CNS concentrations in excess of 62 μg/gram (0.1854 μM) of tissue, and in U87-MG malignant glioma bearing animals, concentrations of 362 μg/gram (1.07 μM) of tumor. FIG. 1C shows untreated and WP1066-treated normal donor peripheral blood mononuclear cells (PBMCs) were assessed for phosphorylated Stat-3 activity (shaded histogram) compared to appropriate isotype controls (clear histogram) by intracellular staining and flow cytometry with a Phospho-Stat3 Alexa Fluor 488 conjugate. FIG. 1D shows 1×10⁶ normal donor PBMCs incubated at 37° C., 5% CO₂ with different doses of WP1066 (0.1 μM-5 μM) for 2 and 4 hours. Cells were stained with fluorescently-labeled anti-CD3 and anti-CD11b antibodies, and propidium iodide (PI) and analyzed by flow cytometry. Macrophages and T cells were gated on CD11b⁺ and CD3⁺ populations, respectively, and cell death (%) was quantified as CD11b⁺PI⁺ or CD3⁺PI⁺ populations in the respective plots. Plots represent the 5 μM maximum dose (clear histogram) compared to control untreated cells (shaded histogram) incubated under similar conditions.

FIG. 2 shows that WP1066 is a powerful immune modulator in glioma patients. Specifically FIG. 1A shows peripheral blood macrophages and glioma-infiltrating microglia/macrophages upregulate costimulatory molecules when incubated with Stat-3 inhibitor WP1066. PBMCs from normal human donors (n=5) were incubated for 24 hrs at 37° C., 5% CO₂, in either medium (RPMI 1640+10% fetal bovine serum) or medium supplemented with 5 μM WP1066. Cells were then stained with fluorescent antibodies to CD11b and either CD80 or CD86 and analyzed by flow cytometry. Glioma-infiltrating microglia were purified from freshly resected GBM patient tumor tissue (n=3) and incubated in either medium or medium supplemented with WP1066 (1 μM). After incubation, microglia (CD11b⁺/CD45⁺) were double stained with fluorescent antibodies to CD11b and either CD80 or CD86, and analyzed by flow cytometry. Numbers in the upper right quadrant of each graph denote the percentage of CD11b⁺ gated cells that express either CD80 or CD86. FIG. 2 shows PBMCs from normal human donors (n=7) were incubated for 4 hrs at 37° C., 5% CO₂, in either medium or medium supplemented with WP1066 (5 μM) or LPS (5 μg/mL). Cells were then labeled with CD11b/CD14 macrophage markers and stained and analyzed for intracellular cytokine production. Percentages of macrophages that were CD11b⁺/CD14⁺ and also positive for the respective cytokine were calculated by flow cytometry analysis. A scatter plot representative of all samples analyzed for macrophage intracellular cytokine production was determined with LPS-treated cells (circles) and WP1066-treated cells (diamonds). The black bars denote the mean values of each subgroup, p values determining statistical significance between LPS-stimulated groups and WP1066-stimulated groups were determined using Student's t test (NS, not statistically significant). FIG. 2C shows WP1066-treated antigen-presenting cells (APCs) can stimulate strong proliferative responses in normally refractive T cells from the peripheral blood of GBM patients. Carboxyfluoroscein succinimidyl ester (CFSE)-labeled CD8⁺ T cells isolated from newly diagnosed GBM patients were incubated with untreated autologous APCs (CD11b⁺/CD14⁺) and anti-CD3 antibody, with anti-CD3 antibody+anti-CD28 antibody, and with LPS-treated (5 μg/mL) autologous APCs or WP1066-treated (1 μM) autologous APCs. In all cases, cells were incubated for 4 days at 37° C., 5% CO₂, followed by surface staining for T cell markers (CD3, CD8) and analysis by flow cytometry. The number on each plot indicates the percentage of CD8⁺ gated cells that have undergone cell division via CFSE dilution. This data is representative of three separate experiments. For (A-C), modified Percoll density gradient (1.083 g/mL) centrifugation was used to isolate PBMCs of both GBM patients and normal donors. CD8+ T cells were isolated by magnetic bead separation and then labeled with CFSE. Autologous APCs were purified from the above density gradient buffy-coat layer by negative selection, using a monocyte bead isolation kit and magnetic separation column. Purity of all cell populations was determined to be at least 97% by flow cytometry. Autologous microglia/macrophages from GBM patients were purified from freshly resected human glioma/brain tissue using a modified Percoll gradient isolation technique. All APCs were treated with the respective stimulants (control medium alone, LPS, or WP1066) for 24 hours and then washed prior to incubation with T cells in the proliferation assay. FIG. 2D shows PBMCs from GBM patients were purified as described in FIG. 2C. T cells from the same patients were isolated with human CD3 microbeads and a magnetic separation column, with the exception of one patient who lacked sufficient PBMCs for both monocyte and T cell isolations. Monocytes or T cells seeded at a density of 1×10⁶ cells per well in 6-well culture plates and were incubated at 37° C., 5% CO₂, with either the medium or medium supplemented with 5 μM WP1066. After 2 hours, monocytes and T cells were stimulated for 5 minutes with 2 μg/mL LPS and 5 μg/mL anti-CD3 antibody, respectively, in the presence or absence of WP1066. Subsequently, cells were lysed in buffer containing 1% Triton-X and protease inhibitors. Protein aliquots (20 μg) from each monocyte and T-cell lysate were electrophoretically fractionated in 8% SDS-polyacrylamide gels, electrophoretically transferred to nitrocellulose membranes, and immunoblotted with antiphosphotyrosine monoclonal antibody 4G10. Autoradiography of the membranes was performed using enhanced chemiluminescence reagents. After stripping the membrane, it was reblotted with antibody to phospho-p72Syk (Tyr352). With subsequent re-stripping, the membrane was re-blotted with antibodies to Syk, phospho-Hck, Hck, and Lyn, respectively. For the T cell membranes, after stripping, the membrane was reblotted with antibody to phospho-ZAP-70 (Tyr319). With subsequent re-stripping, the membrane was reblotted with antibodies to ZAP-70, p56-Lck, and β-Actin, respectively.

Several immunotherapeutic clinical trials in glioma patients have shown promise, but in patients with advanced cancers and grossly evident disease, the objective response rates have remained low. Rosenberg S. A. et al. Nat Med 10, 909-915 (2004). Potent immune activators are necessary to counteract the immunosuppressive factors that can overwhelm an induced immune response. Small molecule inhibitors with well characterized mechanisms of immune modulation, such as WP1066, and similar tyrphostin and tyrphostin-like compounds can be effectively and efficiently used in the setting of immunotherapy and/or vaccine administration.

### Example 2

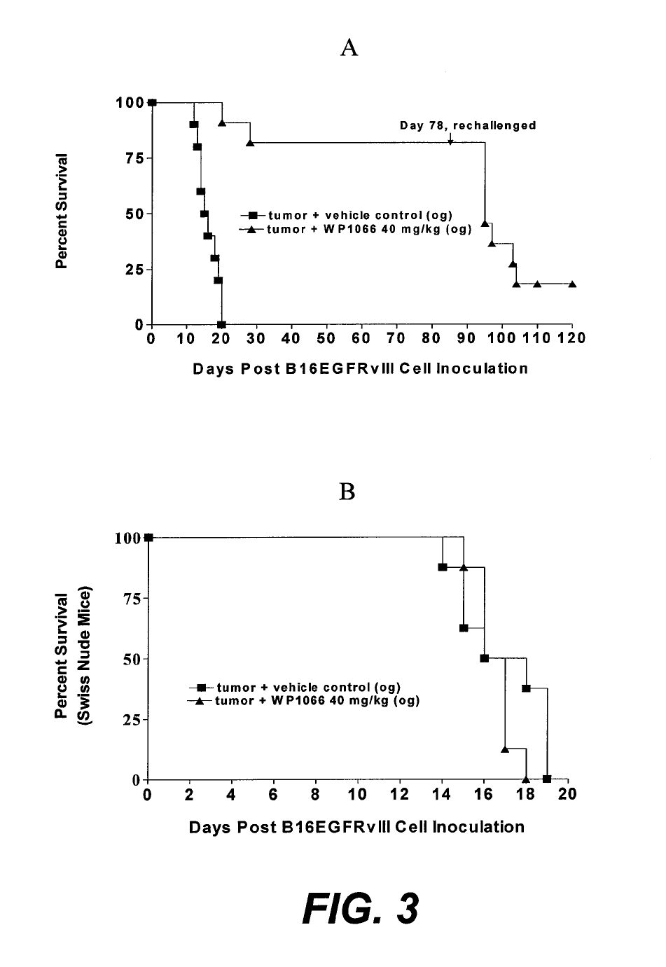

FIG. 3A shows survival data from C57BL/6J mice treated with WP1066 after intracerebral B16EGFRvIII cells were established in the brain. Identical anti-tumor efficacy was also observed in C57BL/6J mice with established intracerebral B16 cells treated with WP1066 via oral gavage (n=10). In animals that survived longer than 78 days, subsequent rechallenge by injection of tumor cells into the contralateral hemisphere indicated that minimal immunological memory was induced.

FIG. 3B shows survival data from nude mice treated with WP1066 after intracerebral B16 were established in the brain. The immune incompetent background abrogates the clinical effectiveness of WP1066. In vivo depletions of the CD4+ and CD8+ T cells abrogates the efficacy of WP1066 in established intracerebral syngeneic murine models, providing further evidence that the immune system is mediating tumor clearance.

### Example 3

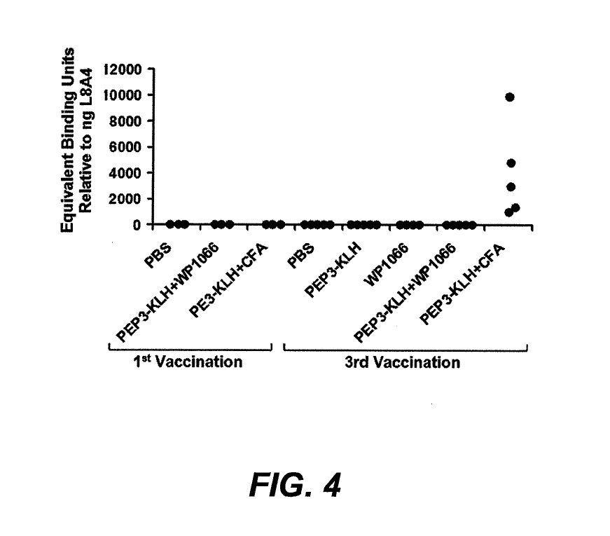

FIG. 4 shows that WP1066 enhances immune cytotoxicity but not humoral responses. Humoral responses were not induced in mice vaccinated with PEP-3-KLH plus WP1066 but were in those vaccinated with the positive control, PEP-3-KLH plus CFA.

### Example 4

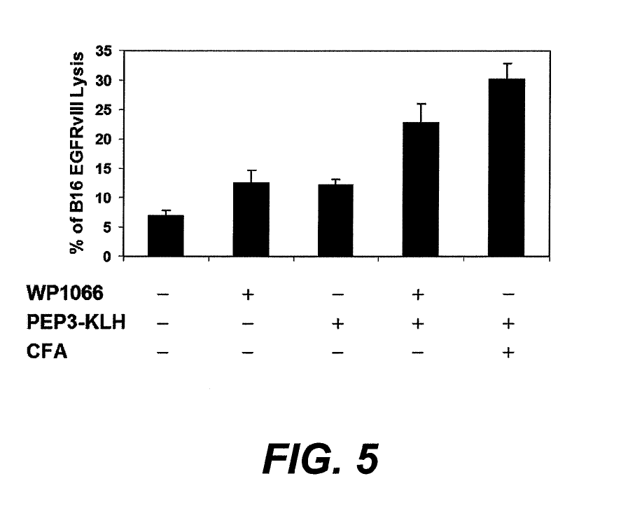

FIG. 5 shows that WP1066 enhances immune cytotoxicity but not humoral responses. Cytotoxicity against the B16EGFRvIII cells in vitro by splenocytes obtained from mice vaccinated with PEP-3-KLH plus WP1066 and compared with that in naïve, PEP-3-KLH-, and PEP-3-KLH plus CFA-vaccinated mice. B16EGFRvIII cells were labeled with CFSE and then added to wells containing titered C57BL/6J splenocytes (E:T=40:1). The splenocyte effector cells from naïve mice induced minimal lysis. However, splenocyte effector cells from mice vaccinated with PEP-3-KLH and WP1066 enhanced EGFRvIII-specific lysis (p<0.05). The error bars show one standard deviation from mean values.

### Example 5

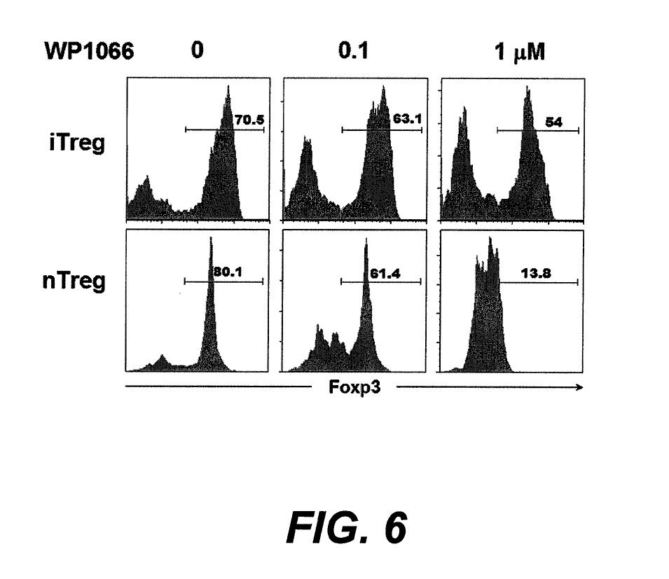

FIG. 6 shows that WP1066 inhibits Foxp3 induction in peripheral T cells and downregulates Foxp3 expression in natural Tregs. CD4+CD25−CD62L^{hi} naïve T cells from C57BL/6J mice were stimulated by plate-bound anti-CD3 (2 μg/ml) and soluble anti-CD28 (2 μg/ml) antibodiesin the presence of TGF-β1 (1 ng/ml) plus hIL-2 (200 U/ml) with addition of different concentrations of WP1066 (0, 0.1, and 1 μM) for inducible Treg (iTreg) differentiation; CD4+CD25+ T cells (natural Tregs [nTreg]) were stimulated by plate-bound anti-CD3 (2 μg/ml) antibodies in the presence of hIL-2 (200 U/ml) with addition of different concentrations of WP1066 (0, 0.1, and 1 μM). More than 96 hours after stimulation, the cells were analyzed for intracellular Foxp3 expression using flow cytometry.

1. A method of treating immunosuppression comprising administering to a subject in need thereof a therapeutic amount of a tryphostin compound of the formula:

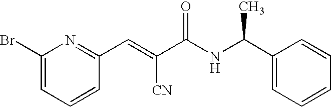

2. A method of inducing immunostimulatory cytokines comprising administering to a subject in need thereof a therapeutic amount of a tryphostin compound of the formula:

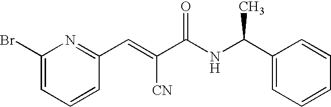

3. A method of inducing costimulatory molecules comprising administering to a subject in need thereof a therapeutic amount of a tryphostin compound of the formula:

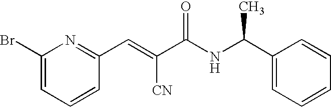

4. A method of enhancing phosphorylation of signaling molecules comprising administering to a subject in need thereof a therapeutic amount of a tryphostin compound of the formula:

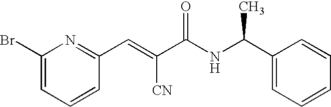

5. A method of enhancing the function of T cells administering to a subject in need thereof a therapeutic amount of a tryphostin compound of the formula: [chemical structure]
